(19) United States
(12) Patent Application Publication
(10) Pub. No.: US 2023/0103033 A1
(43) Pub. Date: Mar. 30, 2023

(54) TWO-PHASED MEDICAL DIAGNOSIS

(71) Applicant: International Business Machines Corporation, Armonk, NY (US)

(72) Inventors: Zhong Fang Yuan, Xi'an (CN); Xiang Yu Yang, Xi'an (CN); Tong Liu, Xi'an (CN); Han Ying Song, Xi'an (CN); Ting LM Li, Xi'an (CN)

(21) Appl. No.: 17/483,847

(22) Filed: Sep. 24, 2021

(51) Int. Cl. G16H 50/20; G16H 30/40; G06F 40/40; G06N 3/08; G06K 9/46

(52) U.S. Cl. CPC G16H 50/20; G16H 30/40; G06F 40/40; G06N 3/08; G06K 9/46

(57) ABSTRACT

Methods, apparatus, computer program products for two-phased medical diagnosis are provided. The computer-implemented method comprises, receiving, by one or more processors, data during a process of a medical diagnosis from a source of data information. The computer-implemented method also comprises extracting, by one or more processors, features from the received data. The computer-implemented method also comprises transferring, by one or more processors, the extracted features in form of feature vectors to a server via a network. The computer-implemented method further comprises obtaining, by one or more processors, a recommendation of medical diagnosis from the server, wherein the recommendation of medical diagnosis is based, at least in part, on labels determined for the feature vectors.

### BACKGROUND

The present invention relates to computer techniques and more particularly, to a method, system, and computer program product for a two-phased medical diagnosis during a process of a health service.

During the process of the health service, on-site medical workers may use terminal devices, such as mobile phones, tablets and so on, to take images, and then send the images to a cloud sever via the Internet to call deep learning algorithms of a neural network for a medical diagnosis. For example, in a scenario of medical diagnosis regarding a skin disease, on-site medical workers may take images of focus areas of skins of patients and send the images to the sever. Then, an on-site diagnosis recommendation based on the deep learning algorithm may be returned to the terminal devices.

### SUMMARY

Embodiments of the present disclosure disclose computer-implemented methods, systems and computer program products. According to some embodiments of the present disclosure, the computer-implemented method comprises, receiving, by one or more processors, data during a process of a medical diagnosis from a source of data information. The computer-implemented method also comprises extracting, by one or more processors, features from the received data. The computer-implemented method also comprises transferring, by one or more processors, the extracted features in form of feature vectors to a server via a network. The computer-implemented method further comprises obtaining, by one or more processors, a recommendation of medical diagnosis from the server, wherein the recommendation of medical diagnosis is based, at least in part, on labels determined for the feature vectors.

The above summary is not intended to describe each illustrated embodiment or every implementation of the present disclosure.

### BRIEF DESCRIPTION OF THE DRAWINGS

FIG. 1 depicts a cloud computing node according to some embodiments of the present disclosure;

FIG. 2 depicts a cloud computing environment according to some embodiments of the present disclosure;

FIG. 3 depicts abstraction model layers according to some embodiments of the present disclosure;

FIG. 4 depicts an example system of the health service;

FIG. 5 depicts an example system of the health service according to embodiments of the present invention;

FIG. 6A depicts an example flow chart of medical diagnosis of a first phase according to embodiments of the present invention;

FIG. 6B depicts an example flow chart of medical diagnosis of a second phase according to embodiments of the present invention; and

FIG. 7 depicts an example flow chart of method for two-phased medical diagnosis according to embodiments of the present invention.

### DETAILED DESCRIPTION

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure. Through the more detailed description of some embodiments of the present disclosure in the accompanying drawings, the above and other objects, features and advantages of the present disclosure will become more apparent, wherein the same reference generally refers to the same components in the embodiments of the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly level out and rapidly released to quickly level in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

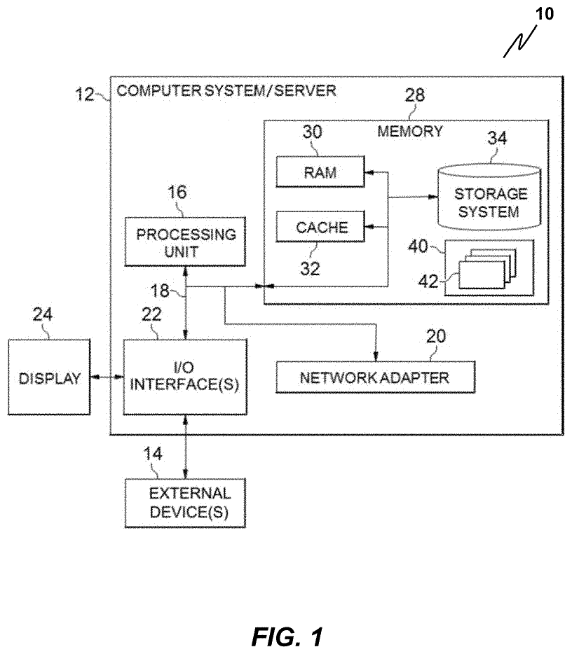

Referring now to FIG. 1, a schematic of an example of a cloud computing node is shown, according to some embodiments of the present disclosure. Cloud computing node 10 is only one example of a suitable cloud computing node and is not intended to suggest any limitation as to the scope of use or functionality of embodiments of the disclosure described herein. Regardless, cloud computing node 10 is capable of implementing and/or performing any of the functionality set forth herein.

In cloud computing node 10 there is a computer system/server 12, which can be a portable electronic device such as a communication device, and/or numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor bus or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM, or other optical media can be provided. In such instances, each can be connected to bus 18 by one or more data media interfaces. As will be further depicted and described below, memory 28 may include at least one program product 40 having a set (e.g., at least one) of program modules 42 that are configured to carry out the functions of embodiments of the disclosure.

Program product 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the disclosure as described herein.

Computer system/server 12 may also communicate with one or more external devices 14 such as a keyboard, a pointing device, and a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

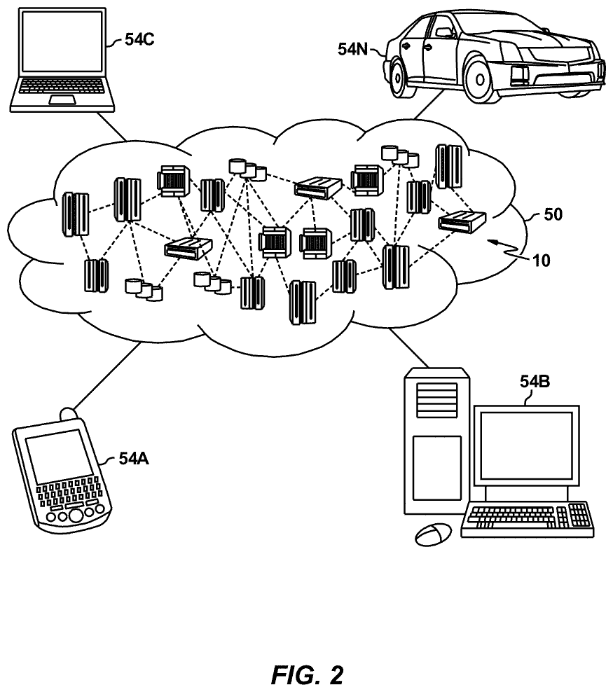

Referring now to FIG. 2, illustrative cloud computing environment 50 is depicted, according to some embodiments of the present disclosure. As shown, cloud computing environment 50 includes one or more cloud computing nodes 10 with which local computing devices used by cloud consumers, such as, for example, personal digital assistant (PDA) or cellular telephone 54A, desktop computer MB, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

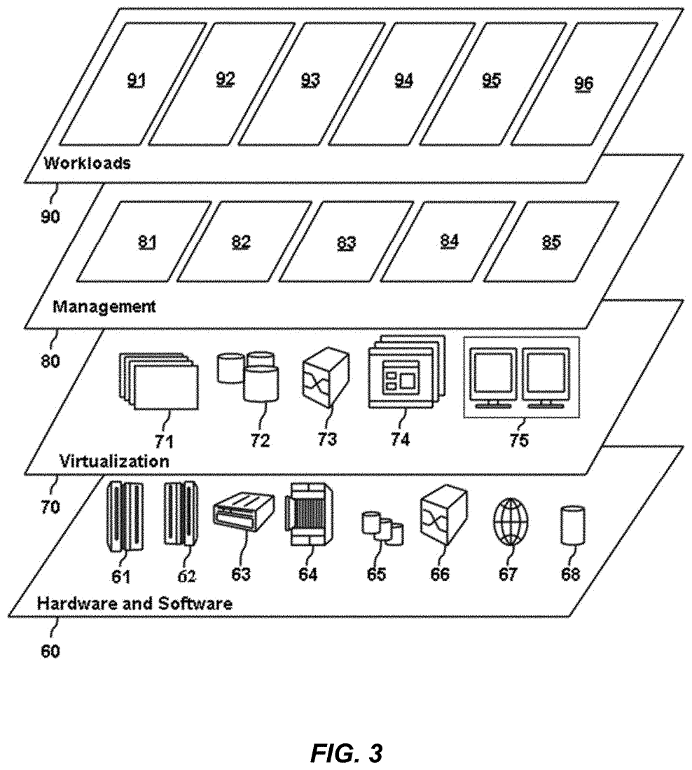

Referring now to FIG. 3, a set of functional abstraction layers provided by cloud computing environment 50 (FIG. 2) is shown, according to some embodiments of the present disclosure. It should be understood in advance that the components, layers, and functions shown in FIG. 3 are intended to be illustrative only and embodiments of the disclosure are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and medical diagnosis 96. The functionalities of medical diagnosis 96 will be described in the following embodiments of the present invention.

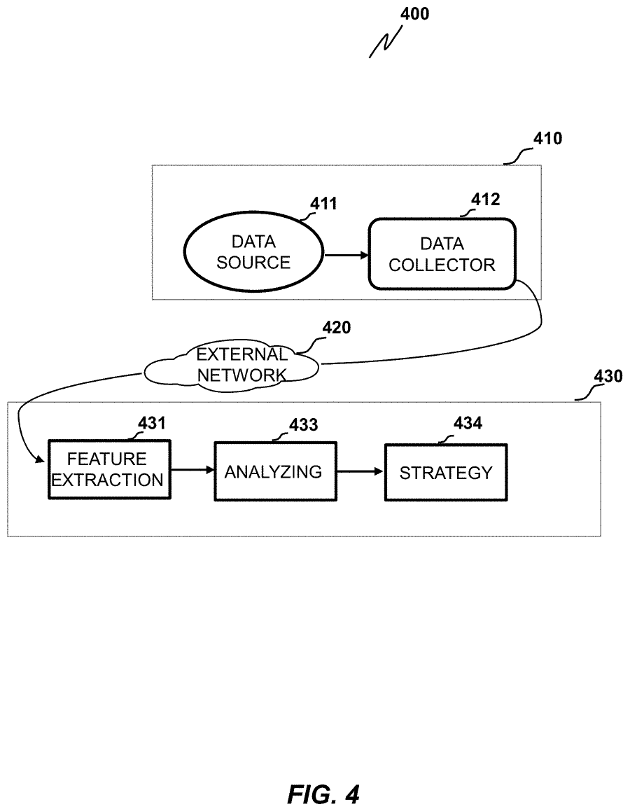

As mentioned above, during the process of the health service, on-site medical workers may use terminal devices to take images, then send the images to the cloud sever via internet to call deep learning algorithm of the neural network for a medical diagnosis. FIG. 4 depicts an example system of the health service 400.

Now referring to FIG. 4, the example system of the health service 400 may contain a terminal device 410 and a cloud server side, or cloud server, 430. The terminal device 410 may be connected to the cloud server side 430 via an external network 420. The external network 420 may be, for example, the Internet. At the terminal device 410, a data source 411 may represent a source of data information in a health service site including patients and medical workers. A data collector 412 may receive the data information from the data source 411. The data processing ability of the terminal devices has been greatly improved, but it is still far from that of hardware devices specialized in a deep learning algorithm in the cloud server 430. Thus, in some solutions, the data collector 412 in the terminal device are generally used to take images from the data source 411, which are then sent to the cloud server side 430 through the external network 420 to execute algorithm operations. The cloud server side 430 may comprise a feature extraction component 431, a task component 433 and a strategy component 434. These components may respectively perform deep learning algorithm of neural network to complete the medical diagnosis. After the algorithm operations, the sever may send the diagnosis recommendation to the terminal device in the form of return value, and the terminal device may obtain the diagnosis recommendation. It may be understood that the medical diagnosis process using deep learning algorithms is basically completed on the cloud server 430.

However, in the solutions mentioned above, there are some restrictions, such as network bandwidth and delay. For example, if the network signal is weak, the transmission rate of images may be relatively slow. Though images may be compressed to save network bandwidth, it may cause undesired distortion of the images. On the other hand, the solutions also have concern of data privacy protection. For example, patients do not want their images uploaded in plaintext through the internet.

In addition, in the solutions, computer vision is usually used for medical diagnosis, which is only from the perspective of vision, and relies too much on the static images themselves. For example, images information is processed through target detection, image classification and so on to help determine the location of lesions. In fact, static images are not adequate to figure out the result since many focuses of infections may only be determined by observing the patient's reaction after pressing the location of lesion, chatting with patient and other interactions. Thus, instead of or in addition to static images, dialogues between the medical workers and the patients, response of the patients, and the video of the interaction may be collected and used for medical diagnosis.

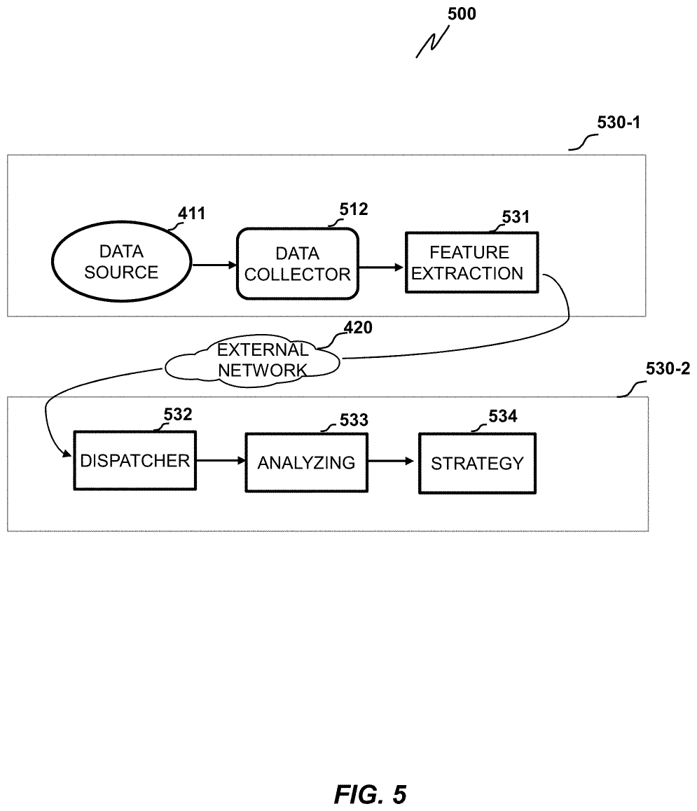

According to example embodiments of the present disclosure, there is proposed a solution for a two-phased medical diagnosis, which cooperates algorithm operations completed in both the terminal device side and the cloud server side to decrease data transfer effort and network delay. FIG. 5 depicts an example system 500 of the health service according to embodiments of the present invention.

In the example system 500, parts of the algorithm operations implemented by the components in the cloud sever shown in FIG. 4 may be migrated to the terminal device 410. That is, the algorithm operation of extracting features from the data source 411 by the feature extraction component 431 (denoted by the reference number 531 in FIG. 5) may be configured in the terminal device 410 (referred to as 530-1 in FIG. 5). The algorithm operation of extracting features by the feature extraction component 531 together with other operations in the terminal device may be referred to as the first phase of medical diagnosis herein. The extracted feature may be transferred to the cloud server, or cloud side, 530 for subsequent algorithm operations to be performed by the dispatcher, or dispatcher component, 532, the task component 533 and the strategy component 534. The subsequent algorithm operations in the cloud side 530 may be referred to as the second phase of the medical diagnosis herein. It would be appreciated that the example system 500 of the health service is merely provided as a specific example, and the number of components depicted in the FIG. 5 is merely shown for the purpose of illustration without implying any limitation. In other examples, a different number of components may work together to provide a similar function or intention.

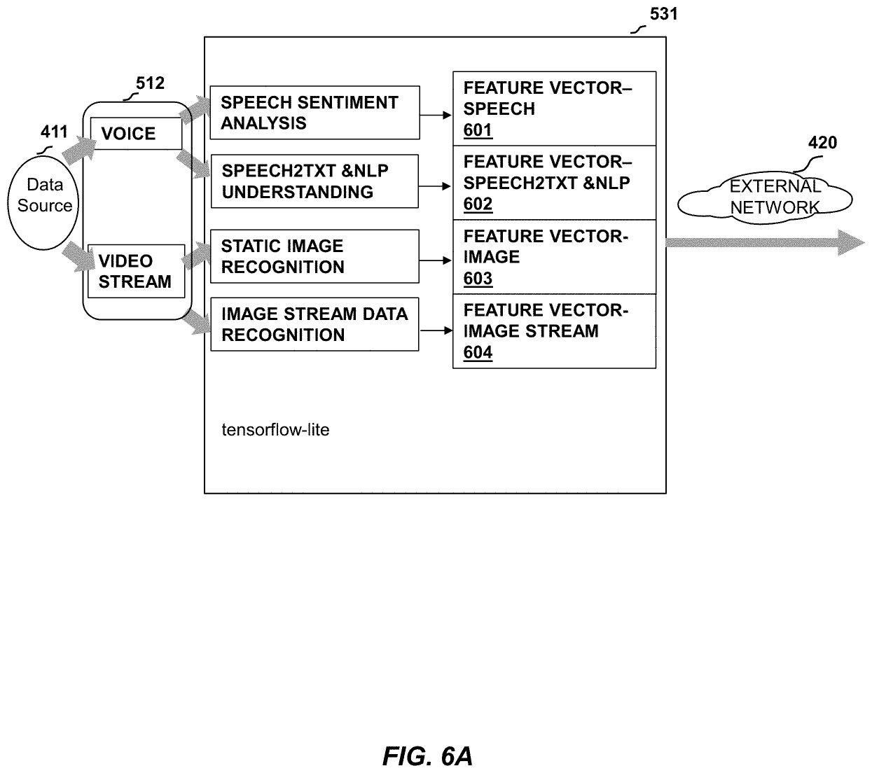

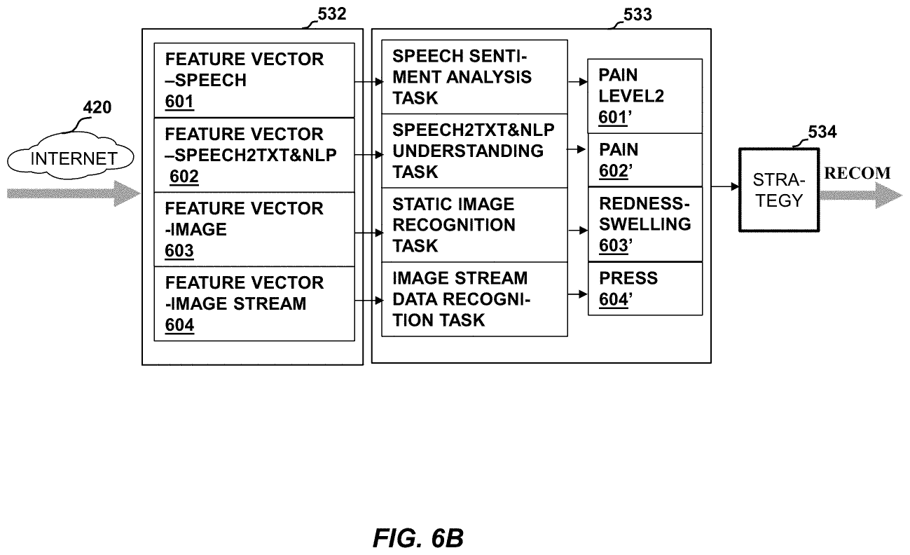

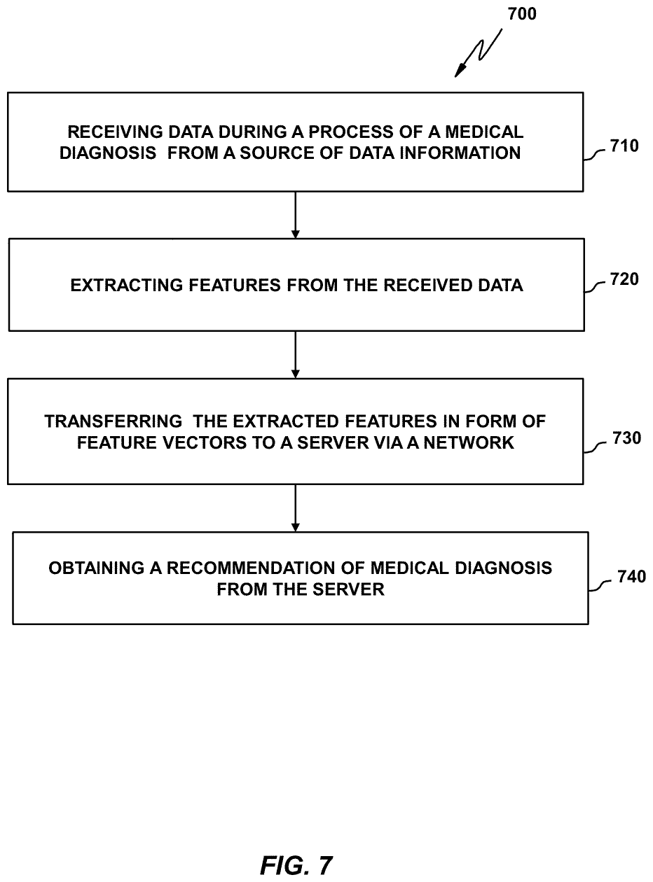

In the two-phased solution, the extracted feature, rather than the images, may be transferred in the form of feature vectors to the cloud server 530 for subsequent algorithm operations. The size of feature vectors is far less than the images, thus data transmission efficiency and network delay may be improved. At the same time, the extracted features may be transmitted in the form of vectors in the network, rather than in the form of plaintext images, which can better protect the privacy of the patients. In addition, a type of data based on which the feature is extracted may no longer be limited to image, but also may include action, NPL, sentiment and so on. For sake of simplification herein, the type of data based on which the feature is extracted is referred to as the type of the feature. The feature may be represented in the form of vector, and the type of the feature may also be referred as the type of the feature vector. Such multiple types of features may facilitate to make an accurate medical diagnosis. The above components in FIG. 5 may be discussed in detail in the following in combination with FIGS. 6-7.

FIG. 6A depicts an example flow chart of the first phase of the medical diagnosis according to embodiments of the present invention. As illustrated, the data collector 512 may collect data information from the data source 411. The data information may comprise video stream with voice. The feature extraction component 531 may receive the video stream with voice and extract features by using feature extraction algorithms. The extracted features may then be transmitted in the form of vectors to the component dispatcher 532 in the cloud sever via external network 420.

As mentioned above, the types of the features may include image, action, NPL, sentiment and so on. The extraction of these types of features may be discussed hereinafter respectively. The feature extraction component 531 may perform a speech sentiment analysis algorithm on voiceprint of the voice to obtain sentiment feature. For example, by using the WaveNet algorithm and obtaining the feature vector “speech” 601. The feature extraction component 531 may perform speech2txt and a natural language processing (NLP) algorithm on the voice to obtain a speech2txt&NPLfeature, for example, by using Seq2Seq algorithm and obtaining the feature vector “speech2txt&NPL” 602. The feature extraction component 531 may perform static image recognition on the video stream to obtain image feature, for example, by using VGG Net algorithm and obtaining the feature vector “image” 603. The feature extraction component 531 may perform image stream data recognition algorithm on the video stream to obtain an action feature, for example, by using OpenPose algorithm and obtaining the feature vector “image stream” 604. The “speech”, the “speech2txt &nlp”, the “image” and the “image stream” may be an identifier for each type of feature vector respectively.

It should be pointed out that the types of feature vectors, the number of each type of feature vector and the identifier for each type of feature vector shown in FIG. 6A are only for illustration. The types of vectors may not be limited to image, action, sentiment and NLP, and all other features contained in the video stream that are beneficial to medical diagnosis may be extracted by the feature extraction component 531. At the same time, there may be more than one feature vector for each type of feature vectors. And any other appropriate identifier for each type of feature vector may be used.

It should also be noted that a framework of TensorFlow-lite may be configured in the feature extraction component 531 and feature extraction algorithms are loaded in the framework. The framework of TensorFlow Lite is a lightweight solution and may be run on terminal devices. The framework is a known technology and will not be discussed in detail herein.

FIG. 6B depicts an example flow chart of the second phase of the medical diagnosis according to embodiments of the present invention. The dispatcher component 532, the task component 533 and the strategy component 534 may belong to a heavy-duty neural network. The dispatcher 532 may dispatch the feature vector to a corresponding algorithm based on the identifier of the feature vector. The task component 533 may be a classification model, which may analyze meaning of each type of feature vector and label the meaning to each type of feature vector. The strategy component 534 may be a diagnosis recommender. The strategy component 534 may also be a classification model. The strategy component 534 may depend on different scenarios, such as matching similar case, medical diagnosis identification, treatment recommendation and so on.

As illustrated in FIG. 6B, the dispatcher 532 may dispatch the feature vector “speech” 601, the feature vector “speech2txt&NLP” 602, the feature vector “image” 603 and the feature vector “image stream” 604 to corresponding algorithm performed by the task component 533. The task component 533 may label the corresponding meaning for each type of feature vector by performing the corresponding algorithm.

According to example embodiments of the present disclosure, the task component 533 may label the corresponding meaning for the feature vector “speech” 601 by performing, for example, the WaveNet algorithm. As mentioned above, the sentiment feature vector may be obtained by using the speech sentiment analysis algorithm on voiceprint of the voice. The voiceprint may contain intonation information, which may be used to analyze the meaning of sentiment of patients. The meaning of the sentiment may include such as “pain-level 1”, “pain-level 2”, “comfort”, etc. The pain-level may indicate the degree of pain of the patient when the medical worker makes an action to a focus of infection. For example, the label for the feature vector “speech” 601 may be “pain-level 2” 601′.

According to example embodiments of the present disclosure, the task component 533 may label the corresponding meaning for the feature vector “speech2txt&NPL” 602 by performing corresponding algorithm. For example, the patient yells “It hurts” during a process of the medical diagnosis in the health service site. The text of “It hurts” may be resolved by using Seq2Seq algorithm by the feature extraction component 531. The meaning “pain” may be obtained by using Seq2Seq and word2vec algorithms by the task component 533. The label for the feature vector “speech2txt &NLP” 602 may be “pain” 602′.

According to example embodiments of the present disclosure, the task component 533 may label the corresponding meaning for the feature vector “image” 603 by performing, for example, the VGG Net algorithm. For example, the label for the feature vector “image” 603 may be “redness and swelling” 603′.

According to example embodiments of the present disclosure, the task component 533 may label the corresponding meaning for the feature vector “image stream” 604 by performing, for example, the OpenPose algorithm. For example, the label for the feature vector “image stream” 604 may be “press” 604′.

According to example embodiments of the present disclosure, the labeled data may be input into the strategy component 534 as input data. The strategy component 534 may find the matched symptoms in a classification library based on the labeled meaning of each type of the feature vector and make the recommendation of a treatment scheme. For example, the recommendation may be “use XXX medicine for three courses”.

It should also be pointed out that the framework and algorithms used herein may be known technologies and will not be discussed in detail. It is also to be understood that the framework and algorithms mentioned herein are only for the purpose of illustration, without suggesting any limitations to the scope of the present disclosure. Instead, any other suitable framework and algorithms currently known or to be developed in the future can be used.

With reference now to FIG. 7, in which an example flow chart of method 700 for two-phased medical diagnosis according to embodiments of the present invention is depicted. The method 700 may comprise operations 710-740.

At operation 710, the data collector 512 may receive data during a process of a medical diagnosis from a source of data information.

At operation 720, the feature extraction component 531 may extract features from the received data.

At operation 730, the feature extraction component 531 may transfer the extracted features in form of feature vectors to the dispatcher 532 via a network 420.

At operation 740, the terminal device 410 may obtain a recommendation of medical diagnosis from the cloud server 430, wherein the recommendation of medical diagnosis is generated by the cloud server 430 through: labeling the feature vectors by the task component 533 in the cloud server 430, and generating the recommendation of medical diagnosis based on the labeled feature vectors by the strategy component 534 in the cloud server 430.

With the operations shown in the FIG. 7, the data transmission efficiency and network delay may be improved. At the same time, the form of feature vectors may better protect the privacy data of the patients than the form of images.

Further, in some embodiments, at operation 710, the data collector 512 may receive the data comprising video streams with voices.

In some embodiments, each of the feature vectors is of a type of at least one of the followings: image, action, sentiment and natural language processing (NLP).

In some embodiments, at operation 740, wherein labeling the feature vectors comprising: the dispatcher component 532 may dispatch each type of feature vector to a corresponding algorithm; and the task component 533 may label meaning for each of the feature vectors based on an analysis using the corresponding algorithm.

In some embodiments, at operation 740, generating the recommendation of medical diagnosis based on the labeled feature vectors comprises: the strategy component 534 generating the recommendation of medical diagnosis based on the labeled meaning of each type of the feature vector.

In some embodiments, the terminal device 410 is configured with a framework of TensorFlow-lite and feature extraction algorithms are loaded in the framework. The feature extraction algorithms comprise at least one of the followings: WaveNet algorithm, Seq2Seq, word2vec algorithm, VGG Net algorithm and OpenPose algorithm.

It should be noted that the processing of two-phased medical diagnosis system according to embodiments of this invention could be implemented by computer system/server 12 of FIG. 1.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

The terminology used herein is for the purpose of describing particular embodiments only and is not intended to be limiting of the various embodiments. As used herein, the singular forms “a,” “an,” and “the” are intended to include the plural forms as well, unless the context clearly indicates otherwise. It will be further understood that the terms “includes” and/or “including,” when used in this specification, specify the presence of the stated features, integers, steps, operations, elements, and/or components, but do not preclude the presence or addition of one or more other features, integers, steps, operations, elements, components, and/or groups thereof. In the previous detailed description of example embodiments of the various embodiments, reference was made to the accompanying drawings (where like numbers represent like elements), which form a part hereof, and in which is shown by way of illustration specific example embodiments in which the various embodiments can be practiced. These embodiments were described in sufficient detail to enable those skilled in the art to practice the embodiments, but other embodiments can be used, and logical, mechanical, electrical, and other changes can be made without departing from the scope of the various embodiments. In the previous description, numerous specific details were set forth to provide a thorough understanding the various embodiments. However, the various embodiments can be practiced without these specific details. In other instances, well-known circuits, structures, and techniques have not been shown in detail in order not to obscure embodiments.

What is claimed is:

1. A computer-implemented method, the method comprising:
receiving, by one or more processors, data during a process of a medical diagnosis from a source of data information;
extracting, by one or more processors, features from the received data;
transferring, by one or more processors, the extracted features in form of feature vectors to a server via a network; and
obtaining, by one or more processors, a recommendation of medical diagnosis from the server, wherein the recommendation of medical diagnosis is based, at least in part, on labels determined for the feature vectors.

2. The computer-implemented method of claim 1, wherein the data comprises video streams with voices.

3. The computer-implemented method of claim 2, wherein each of the feature vectors is of a type selected from the group consisting of: image, action, sentiment, and natural language processing (NLP).

4. The computer-implemented method of claim 3, wherein the labels are based, at least in part, on analysis of the feature vectors by corresponding algorithms.

5. The computer-implemented method of claim 4, wherein the algorithms correspond to different types of feature vectors.

6. The computer-implemented method of claim 1, wherein the one or more processors are configured with a framework of TensorFlow-lite, and wherein feature extraction algorithms are loaded in the framework.

7. The computer-implemented method of claim 6, wherein the feature extraction algorithms are selected from the group consisting of: WaveNet algorithm, seq2seq algorithm, word2vec algorithm, VGG Net algorithm and OpenPose algorithm.

8. A computer-implemented system, comprising:
at least one processing unit; and
a memory coupled to the at least one processing unit and storing instructions thereon, the instructions, when executed by the at least one processing unit, performing actions comprising:
receiving data during a process of a medical diagnosis from a source of data information;
extracting features from the received data;
transferring the extracted features in form of feature vectors to a server via a network; and
obtaining, by one or more processors, a recommendation of medical diagnosis from the server, wherein the recommendation of medical diagnosis is based, at least in part, on labels determined for the feature vectors.

9. The computer-implemented system of claim 8, wherein the data comprises video streams with voices.

10. The computer-implemented system of claim 9, wherein each of the feature vectors is of a type selected from the group consisting of: image, action, sentiment, and natural language processing (NLP).

11. The computer-implemented system of claim 10, wherein the labels are based, at least in part, on analysis of the feature vectors by corresponding algorithms.

12. The computer-implemented system of claim 11, wherein the algorithms correspond to different types of feature vectors.

13. The computer-implemented system of claim 8, wherein the one or more processors are configured with a framework of TensorFlow-lite, and wherein feature extraction algorithms are loaded in the framework.

14. The computer-implemented system of claim 13, wherein the feature extraction algorithms are selected from the group consisting of: WaveNet algorithm, seq2seq algorithm, word2vec algorithm, VGG Net algorithm and OpenPose algorithm.

15. A computer program product comprising a computer readable storage medium having program instructions embodied therewith, the program instructions executable by an electronic device to cause the electronic device to perform actions comprising:
receiving data during a process of a medical diagnosis from a source of data information;
extracting features from the received data;
transferring the extracted features in form of feature vectors to a server via a network; and
obtaining, by one or more processors, a recommendation of medical diagnosis from the server, wherein the recommendation of medical diagnosis is based, at least in part, on labels determined for the feature vectors.

16. The computer program product of claim 15, wherein the data comprises video streams with voices.

17. The computer program product of claim 16, wherein each of the feature vectors is of a type selected from the group consisting of: image, action, sentiment, and natural language processing (NLP).

18. The computer program product of claim 17, wherein the labels are based, at least in part, on analysis of the feature vectors by corresponding algorithms.

19. The computer program product of claim 18, wherein the algorithms correspond to different types of feature vectors.

20. The computer program product of claim 15, wherein the one or more processors are configured with a framework of TensorFlow-lite, and wherein feature extraction algorithms are loaded in the framework.
